(19) United States
(12) Patent Application Publication
(10) Pub. No.: US 2015/0027209 A1
(43) Pub. Date: Jan. 29, 2015

(54) SENSOR

(71) Applicant: AISAN KOGYO KABUSHIKI KAISHA, Aichi-ken (JP)

(72) Inventor: Masaki Ikeya, Aichi-ken (JP)

(21) Appl. No.: 13/950,219

(22) Filed: Jul. 24, 2013

(51) Int. Cl. G01N 27/22; G01N 33/22

(52) U.S. Cl. CPC G01N 27/226; G01N 33/225
USPC 73/61.61

(57) ABSTRACT

A sensor may comprise a first tubular electrode and a second electrode. The second electrode may be disposed within the first electrode so as to face an inner circumferential surface of the first electrode. A slit may be formed on one of or both of the first and second electrodes so as to communicate between an inside and an outside of the electrode on which the slit is formed over an entire length of a range where the electrode on which the slit is formed faces the other electrode in an axial direction of the first electrode.

### CROSS-REFERENCE TO RELATED APPLICATION

This application claims priority to Japanese Patent Application No. 2012-164372 filed on Jul. 25, 2012, the contents of which are hereby incorporated by reference into the present application.

### TECHNICAL FIELD

The technique disclosed in the present description relates to a sensor that includes a tubular electrode and an electrode disposed within the tubular electrode.

### DESCRIPTION OF RELATED ART

Japanese Patent Application Publication No. 2011-164085 discloses a concentration detection device that detects a concentration of alcohol in fuel stored in a fuel tank. The concentration detection device includes a cylindrical outer electrode and an inner electrode accommodated in the outer electrode. The outer electrode has an opening through which fuel can pass. The opening includes a first opening and a second opening formed at intermediate positions of a side wall of the outer electrode and a third opening formed close to the distal end of the outer electrode. The fuel flows into a gap between the outer electrode and the inner electrode through any of the first to third openings and flows out of the outer electrode through any of the first to third openings. The concentration detection device detects the concentration of alcohol in the fuel using a capacitance between the outer electrode and the inner electrode.

### SUMMARY

In the above configuration, the fuel flows into the gap between the outer electrode and the inner electrode from any of the first to third openings toward the other of the first to third openings. Since the first and second openings are formed at the intermediate positions of the side wall of the outer electrode, there is a case where the fuel present in the gap between the outer electrode and the inner electrode does not flow smoothly in a region where the openings are not formed (that is, in a region above or below the openings). In this case, even when new fuel having different alcohol concentration is stored in the fuel tank, old fuel remains in the gap between the outer electrode and the inner electrode, and it is difficult to appropriately detect the alcohol concentration of the new fuel.

The present description provides a technique for allowing liquid present in a gap between a first tubular electrode and a second electrode disposed inside the first electrode to flow smoothly.

The present application discloses a sensor. The sensor may comprise a first tubular electrode and a second electrode disposed within the first electrode so as to face an inner circumferential surface of the first electrode. A slit may be formed on one of or both of the first and second electrodes so as to communicate between an inside and an outside of the electrode on which the slit is formed over an entire length of a range where the electrode on which the slit is formed faces the other electrode in an axial direction of the first electrode.

When the sensor is immersed in liquid, the liquid flows into the gap between the first and second electrodes. The liquid present in the gap between the first and second electrodes passes through the slit and flows out of the gap between the first and second electrodes. The slit is formed over the entire length in the range where one of the first and second electrodes on which the slit is formed faces the other electrode in the axial direction of the first electrode. According to this configuration, the liquid may suppressed from being stagnated in the gap between the first and second electrodes. As a result, the liquid in the gap between the first and second electrodes may smoothly flow.

### BRIEF DESCRIPTION OF DRAWINGS

FIG. 1 shows a schematic longitudinal cross-sectional view of a sensor system according to a first embodiment.

FIG. 2 shows a II-II cross section of FIG. 1.

FIG. 3 shows a schematic longitudinal cross-sectional view of a sensor system according to a second embodiment.

FIG. 4 shows a schematic longitudinal cross-sectional view of a sensor system according to a third embodiment.

FIG. 5 shows a V-V cross section of FIG. 4.

FIG. 6 shows a schematic longitudinal cross-sectional view of a sensor system according to a fourth embodiment.

FIG. 7 shows a VII-VII cross section of FIG. 6.

FIG. 8 shows a schematic longitudinal cross-sectional view of a sensor system according to a fifth embodiment.

FIG. 9 shows a IX-IX cross section of FIG. 8.

FIG. 10 shows a schematic longitudinal cross-sectional view of a sensor system according to a sixth embodiment.

FIG. 11 shows a XI-XI cross section of FIG. 10.

### DETAILED DESCRIPTION

Some of the features of embodiments in which a sensor includes a first tubular electrode and a second electrode, one or both of which having a slit formed thereon disclosed herein will be listed.

The second electrode may have a tubular shape. The slit may be formed on each of the first and second electrodes, and the slit may include a first slit and a second slit. The first slit may be formed on the first electrode over an entire length of a range where the first electrode faces the second electrode in an axial direction of the first electrode. The second slit may be formed on the second electrode over an entire length of a range where the second electrode faces the first electrode in the axial direction of the first electrode. The second slit may be formed at such a position that the second slit does not face the first slit. In this configuration, the fuel may flow into the gap between the first and second electrodes through any one of the first and second slits and flow out of the gap between the first and second electrodes after passing through the other one of the first and second slits. The second slit is formed at such a position that the second slit does not face the first slit. Thus, the fuel having flowed into the gap between the first and second electrodes flows at least through the gap between the position where the first slit is formed and the position where the second slit is formed. According to this configuration, the liquid may smoothly flow through the gap between the first and second electrodes as compared to a configuration in which the first and second slits are formed at such positions where both slits face each other.

The second electrode may have a tubular shape. The sensor may further include a third tubular electrode disposed within the second electrode so as to face an inner circumferential surface of the second electrode. A third slit may be formed on the third electrode over an entire length of a range where the third electrode faces the second electrode in the axial direction of the first electrode. According to this configuration, a capacitance of the sensor may be increased.

The sensor may further comprise a terminal connected to the second electrode. The slit may be formed on the first electrode over an entire length of a range where the first electrode faces the second electrode in the axial direction of the first electrode. The terminal may pass through the slit from the outside of the first electrode and be connected to the second electrode. According to this configuration, the terminal portion may pass through the slit to make contact with the second electrode from the outer side of the first electrode. Thus, it is not necessary to separately provide a configuration such as a hole for connecting the second electrode and the terminal portion.

The sensor may further comprise a fixture that fixes the second electrode to the first electrode. The slit may be formed on the first electrode over an entire length of a range where the first electrode faces the second electrode in the axial direction of the first electrode. The fixture may pass through the slit from the outside of the first electrode and makes contact with the second electrode. According to this configuration, the fixture may pass through the slit to make contact with the second electrode from the outer side of the first electrode. Thus, it is not necessary to separately provide a configuration such as a hole for connecting the second electrode and the fixture.

The sensor may further comprise a fixture attached to both ends of the first and second electrodes in the axial direction so as to fix the second electrode to the first electrode. According to this configuration, the first and second electrodes may be supported at their both ends in the axial direction. As a result, a distance between the first and second electrodes may be maintained with high accuracy.

The sensor may further comprise a terminal connected to the first and second electrodes. The terminal may pass through the fixture from a surface of the fixture opposite to the first electrode and be connected to the first and second electrodes. According to this configuration, the fixture may support the terminal portion.

The second electrode may have a tubular shape. The sensor may further include a temperature sensor supported by the fixture and accommodated inside the second electrode. According to this configuration, the sensor may detect a liquid temperature.

Representative, non-limiting examples of the present invention will now be described in further detail with reference to the attached drawings. This detailed description is merely intended to teach a person of skill in the art further details for practicing preferred aspects of the present teachings and is not intended to limit the scope of the invention. Furthermore, each of the additional features and teachings disclosed below may be utilized separately or in conjunction with other features and teachings to provide improved sensors, as well as methods for using and manufacturing the same.

Moreover, combinations of features and steps disclosed in the following detailed description may not be necessary to practice the invention in the broadest sense, and are instead taught merely to particularly describe representative examples of the invention. Furthermore, various features of the above-described and below-described representative examples, as well as the various independent and dependent claims, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

### First Embodiment

### Configuration of Sensor System 2

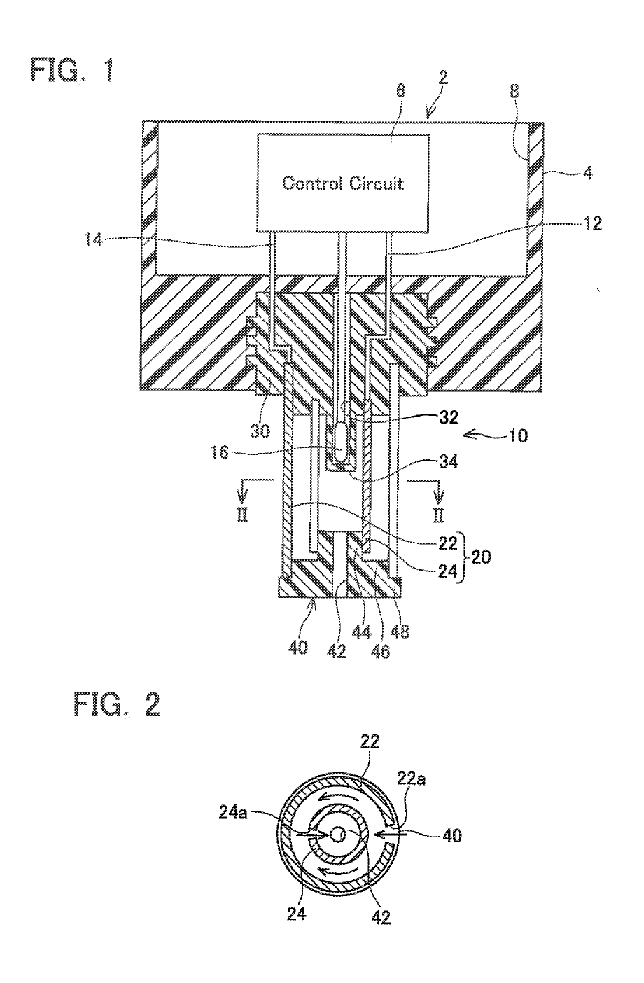

A sensor system 2 shown in FIG. 1 is mounted on a vehicle. The sensor system 2 is used for specifying a concentration of ethanol included in fuel in a fuel tank (not shown) such as a fuel supply system (a fuel tank, a fuel pipe, a delivery pipe, and the like; not shown) extending from the fuel tank to an engine or a return pipe (not shown) for returning surplus fuel from the fuel pump to the fuel tank or the like. The sensor system 2 includes a circuit accommodating unit 4, a control circuit 6, and a sensor 10.

The circuit accommodating unit 4 is attached to a lid of the fuel tank, a fuel pipe, a delivery pipe, or the like. The circuit accommodating unit 4 is formed of resin. The circuit accommodating unit 4 includes a circuit accommodating chamber 8 that accommodates the control circuit 6.

The control circuit 6 is a circuit for controlling the sensor 10. The control circuit 6 supplies a signal (for example, an AC voltage of 10 Hz to 3 MHz) supplied from an oscillation circuit (not shown) to the sensor 10. The control circuit 6 specifies the concentration of ethanol included in fuel using the sensor 10 and outputs the concentration to an external device (for example, engine control unit (ECU)).

The sensor 10 includes a pair of electrodes 20, two terminals 12 and 14, a thermistor 16, and fixtures 30 and 40. In FIG. 1, the pair of electrodes 20 includes two electrodes 22 and 24. The electrode 22 has a cylindrical shape. As shown in FIG. 2, the electrode 22 has a slit 22a that extends in parallel to an axial direction of the electrode 22. The slit 22a extends from one end (upper end) of the electrode 22 to the other end (lower end). That is, the electrode 22 has a circular-arc shape (C-shape) in which a portion where the slit 22a is formed is cut when seen in a cross-section perpendicular to the axial direction.

The electrode 24 is disposed inside the electrode 22. The electrode 24 is disposed at a predetermined distance from an inner circumferential surface of the electrode 22. The electrode 24 has a cylindrical shape. The axial direction of the electrode 24 is parallel to the axial direction of the electrode 22. An outer circumferential surface of the electrode 24 faces the inner circumferential surface of the electrode 22. A length in the axial direction of the electrode 24 is smaller than a length in the axial direction of the electrode 22. The electrode 24 is disposed on the inner side than both the upper and lower ends of the electrode 22. The electrode 24 has a slit 24a that extends in parallel to the axial direction (that is, the axial direction of the electrode 24) of the electrode 22. The slit 24a extends from the upper end of the electrode 24 to the lower end. The slit 24a is formed at a position where the slit 24a does not face the slit 22a. In other words, a direction extending from an axial center of the electrodes 22 and 24 to a central position of a width (the length in the circumferential direction of the electrode 22) of the slit 22a is different from a direction extending from the axial center of the electrodes 22 and 22 to a central position of a width (the length in the circumferential direction of the electrode 24) of the slit 24a. Specifically, the slit 24a is formed on an opposite side (that is, at a position rotated by 180°) from the slit 22a with the axial center interposed. The electrode 24 has a circular-arc shape (C-shape) in which a portion where the slit 24a is formed is cut when seen in the cross-section perpendicular to the axial direction.

As shown in FIG. 1, the fixtures 30 and 40 that fix the electrodes 22 and 24 are disposed at both upper and lower ends of the electrodes 22 and 24. The fixture 30 covers part of the upper ends of the electrodes 22 and 24. The fixture 30 is supported by the circuit accommodating unit 4. The terminals 12 and 14 are disposed in the fixture 30. The terminal 12 extends from the upper end of the electrode 24 to the circuit accommodating chamber 8 while passing through the fixture 30. One end (upper end) of the terminal 12 is in contact with the control circuit 6, and the other end (lower end) of the terminal 12 is in contact with the electrode 24. The terminal 14 extends from the upper end of the electrode 24 to the circuit accommodating chamber 8 while passing through the fixture 30. One end (upper end) of the terminal 14 is in contact with the control circuit 6, and the other end (lower end) of the terminal 14 is in contact with the electrode 22. Since the terminals 12 and 14 are covered with the fixture 30, it is possible to suppress fuel from entering into the circuit accommodating chamber 8 along the terminals 12 and 14. Moreover, the terminals 12 and 14 are disposed in a curved state. According to this configuration, it is possible to further suppress fuel from entering into the circuit accommodating chamber 8 along the terminals 12 and 14.

An accommodating portion 34 in which the thermistor 16 is accommodated is provided in the fixture 30. The accommodating portion 34 communicates with the circuit accommodating chamber 8 through an insertion hole 32. The accommodating portion 34 protrudes toward the inside of the electrode 24. The thermistor 16 is connected to the control circuit 6. The thermistor 16 is disposed inside the electrode 24.

The fixture 40 is in contact with the lower ends of the electrodes 22 and 24. The fixture 40 includes a base 48, an intermediate portion 46, and an upper portion 44. The base 48, the intermediate portion 46, and the upper portion 44 have a cylindrical shape. The lower end of the electrode 22 is in contact with the upper surface of the base 48. The electrode 24 is not in contact with the base 48. The intermediate portion 46 protrudes upward from the upper surface of the base 48. The intermediate portion 46 makes contact with the inner circumferential surface of the electrode 22. The upper portion 44 protrudes upward from the upper surface of the intermediate portion 46. The upper portion 44 makes contact with the inner circumferential surface of the electrode 24. In a modification, the fixture 40 may support the electrodes 22 and 24 by making contact with at least one of the inner circumferential surfaces and the outer circumferential surfaces of the electrodes 22 and 24.

Both upper and lower ends of the electrodes 22 and 24 are fixed by the fixtures 30 and 40. According to this configuration, it is possible to cause the electrode 24 to be strongly aligned with respect to the electrode 22. As a result, it is possible to prevent a change in the capacitance of the pair of electrodes 20 due to a change in the distance between the electrodes 22 and 24.

The fixture 40 includes a through-hole 42 formed at the axial center of the base 48, the intermediate portion 46, and the upper portion 44. The through-hole 42 passes from the upper end of the fixture 40 to the lower end and communicates the outside of the sensor 10 with the inside of the electrode 24.

As shown in FIG. 2, the fuel passes through the slit 22a and flows into the gap between the electrodes 22 and 24. In FIG. 2, the flow of fuel is indicated by an arrow. The same is true for FIGS. 5, 7, 9, and 11. The fuel having flowed into the gap between the electrodes 22 and 24 branches into two along the outer wall of the electrode 24 and flows through the gap between the electrodes 22 and 24. The fuel flows from the slit 22a to the slit 24a through the gap between the electrodes 22 and 24. The slit 24a is formed at a position rotated by 180° about the axial center of the electrodes 22 and 24 with respect to the slit 22a. According to this configuration, it is possible to prevent a region, in which fuel does not flow smoothly, from being formed in the gap between the electrodes 22 and 24 in the circumferential direction (that is, a direction vertical to the axial direction) of the electrodes 22 and 24. Moreover, the slits 22a and 24a are formed over the entire length in the axial direction of the electrodes 22 and 24. Thus, it is possible to prevent a region, in which fuel does not flow smoothly, from being formed in the gap between the electrodes 22 and 24 in the axial direction of the electrodes 22 and 24. According to this configuration, it is possible to prevent fuel from being stagnated in the gap between the electrodes 22 and 24 so that the fuel does not flow smoothly in the gap. The fuel flowing into the electrode 24 through the slit 24a flows outside the sensor 10 by passing through the through-hole 42.

The control circuit 6 specifies the ethanol concentration in the fuel using the capacitance of the pair of electrodes 20, the temperature of the fuel specified by the thermistor 16, and a database stored in advance in a memory (not shown) of the sensor system 2.

### Second Embodiment

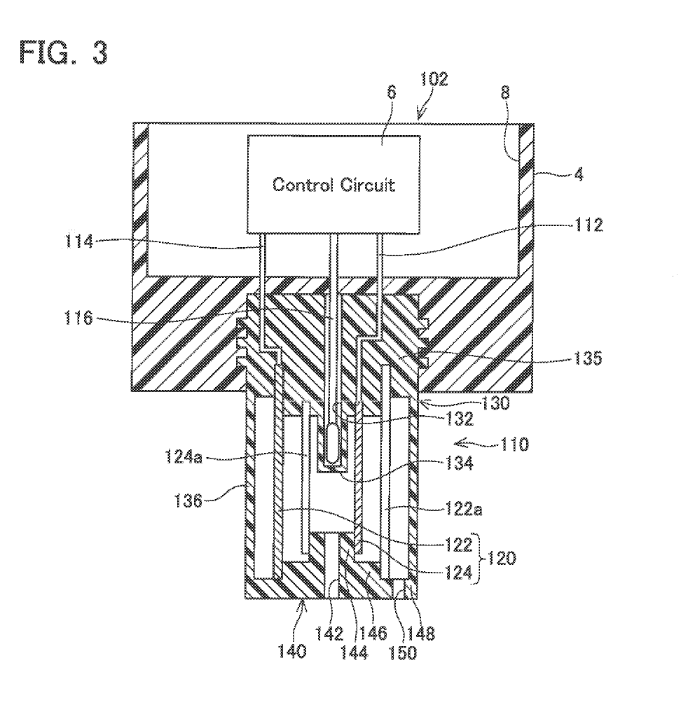

As shown in FIG. 3, a sensor system 102 of a second embodiment includes a circuit accommodating unit 4, a control circuit 6, and a sensor 110. The circuit accommodating unit 4 and the control circuit 6 have the same configuration as those of the first embodiment, and description thereof will not be provided.

The sensor 110 includes a pair of electrodes 120, two terminals 112 and 114, a thermistor 116, and a fixture 130. The pair of electrodes 120 includes two electrodes 122 and 124. The electrodes 122 and 124 have the same configuration as the electrodes 22 and 24, respectively, of the first embodiment, and description thereof will not be provided.

The fixture 130 is supported by the circuit accommodating unit 4. The fixture 130 is formed of resin. The fixture 130 accommodates the pair of electrodes 120. The fixture 130 includes an upper end fixing portion 135, a circumferential wall 136, and a lower end fixing portion 140. The upper end fixing portion 135 covers part of the upper ends of the electrodes 122 and 124. The terminals 112 and 114 are disposed in the upper end fixing portion 135. The terminals 112 and 114 have the same configuration as the terminals 12 and 14, respectively, and description thereof will not be provided. The upper end fixing portion 135 includes an accommodating portion 134 in which the thermistor 116 is accommodated. The accommodating portion 134 communicates with the circuit accommodating chamber 8 through an insertion hole 132. The accommodating portion 134 protrudes toward the inside of the electrode 124. The thermistor 116 is connected to the control circuit 6. The thermistor 116 is disposed inside the electrode 124.

A cylindrical circumferential wall 136 that extends downward is provided on the lower surface of the upper end fixing portion 135. The circumferential wall 136 extends from the lower surface of the upper end fixing portion 135 up to the same height as the lower end of the electrode 122. The lower end fixing portion 140 is fixed to the lower end of the circumferential wall 136. The upper end of the cylindrical circumferential wall 136 is blocked by the upper end fixing portion 135, and the lower end of the cylindrical circumferential wall 136 is blocked by the lower end fixing portion 140.

The lower end fixing portion 140 is in contact with the lower ends of the electrodes 122 and 124. The lower end fixing portion 140 includes a base 148, an intermediate portion 146, and an upper portion 144. The intermediate portion 146 and the upper portion 144 have the same configuration as the intermediate portion 46 and the upper portion 44, respectively, of the first embodiment. The lower end of the electrode 122 is in contact with the upper surface of the base 148. The electrode 124 is not in contact with the base 148. A through-hole 150 that communicates with the gap between the electrode 122 and the circumferential wall 136 is formed on the base 148. A center of the through-hole 150 is positioned on a straight line that connects an axial center and a center in a width direction of the slit 122a when seen in a cross-section perpendicular to an axial direction of the electrodes 122 and 124. The through-hole 142 has the same configuration as the through-hole 42.

The through-hole 142 is connected to a fuel pump (not shown). As a result, the fuel pressurized by the fuel pump flows into the sensor 110 through the through-hole 142 and is discharged from the through-hole 150. According to this configuration, the sensor 110 can provide the same advantages as the sensor 10 of the first embodiment.

### Third Embodiment

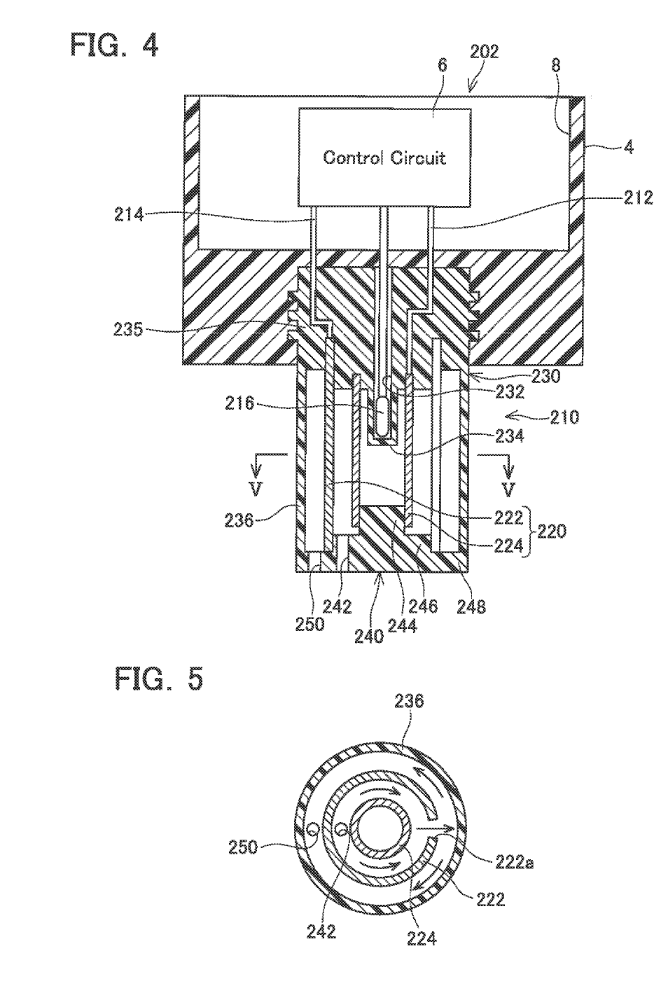

As shown in FIG. 4, a sensor system 202 of a third embodiment includes a circuit accommodating unit 4, a control circuit 6, and a sensor 210. The circuit accommodating unit 4 and the control circuit 6 have the same configuration as those of the first embodiment, and description thereof will not be provided.

The sensor 210 includes a pair of electrodes 220, two terminals 212 and 214, a thermistor 216, and a fixture 230. The pair of electrodes 220 includes two electrodes 222 and 224. The electrode 222 has the same configuration as the electrode 22 of the first embodiment, and description thereof will not be provided.

The electrode 224 is disposed inside the electrode 222. The electrode 224 is the same as the electrode 24 except that the slit 24a is not formed. That is, the electrode 224 is a cylinder in which no cut is formed in a circumferential direction.

The fixture 230 is supported by the circuit accommodating unit 4. The fixture 230 is formed of resin. The fixture 230 accommodates the pair of electrodes 220. The fixture 230 includes an upper end fixing portion 235, a circumferential wall 236, and a lower end fixing portion 240. The upper end fixing portion 235 and the circumferential wall 236 have the same configuration as the upper end fixing portion 135 and the circumferential wall 136, respectively, of the first embodiment, and description thereof will not be provided. Moreover, the terminals 212 and 214 have the same configuration as the terminals 12 and 14, respectively, and description thereof will not be provided. The thermistor 216 is disposed in the accommodating portion 234 and communicates with the circuit accommodating chamber 8 through the insertion hole 232 similarly to the thermistor 116.

The lower end fixing portion 240 is fixed to the lower end of the circumferential wall 236. The lower end fixing portion 240 has the same configuration as the lower end fixing portion 140 except that the positions of the through-holes 242 and 250 are different from the positions of the through-holes 142 and 150. The through-hole 242 communicates a gap between the electrodes 222 and 224 with an outside of the sensor 210. As shown in FIG. 5, the through-hole 242 is formed on an opposite side (that is, at a position rotated by 180°) from the slit 222a of the electrode 222 with an axial center of the electrodes 222 and 224 interposed. The through-hole 250 is formed on an opposite side (that is, at a position rotated by 180°) from the slit 222a with the axial center of the electrodes 222 and 224 interposed.

The through-hole 242 is connected to a fuel pump (not shown). As a result, the fuel pressurized by the fuel pump flows into the sensor 210 through the through-hole 242 and is discharged from the through-hole 250. The fuel flowing into the gap between the electrodes 222 and 224 flows from the through-hole 242 toward the slit 222a. The through-hole 242 is formed at a position rotated by 180° from the slit 222a with the axial center of the electrodes 222 and 224 interposed. Thus, the fuel flows smoothly over the entire range of the gap between the electrodes 222 and 224. According to this configuration, the sensor 210 can provide the same advantages as the sensor 10 of the first embodiment. Moreover, the slit 222a is formed at a position rotated by 180° from the through-hole 250 with the axial center of the electrodes 222 and 224 interposed. Thus, the fuel flows smoothly over the entire range of the gap between the electrode 222 and the circumferential wall 236.

### Fourth Embodiment

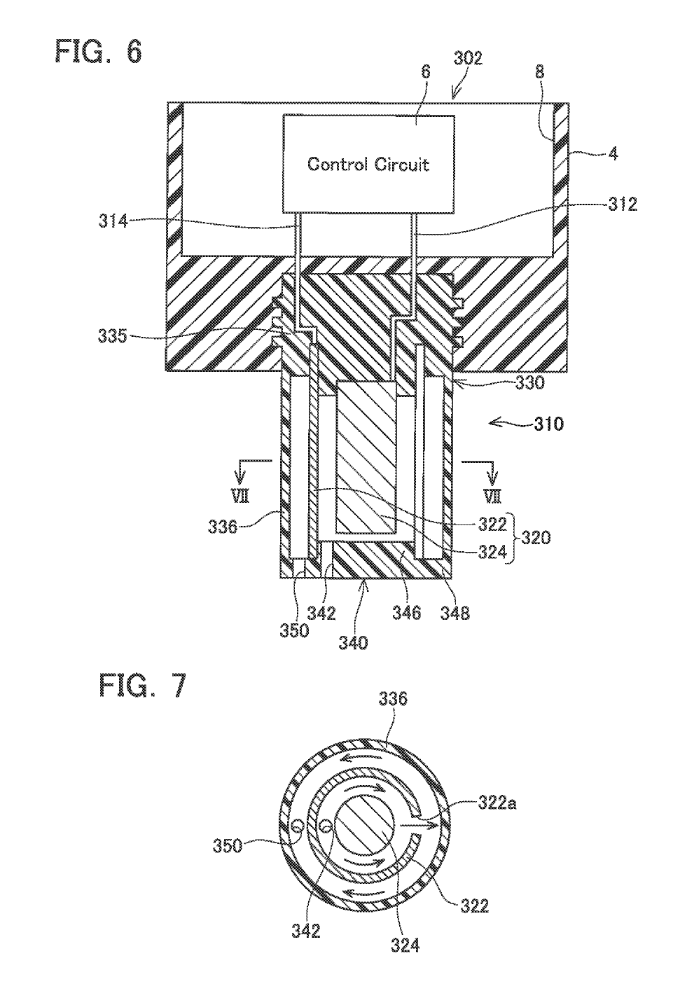

As shown in FIG. 6, a sensor system 302 of a fourth embodiment includes a circuit accommodating unit 4, a control circuit 6, and a sensor 310. The circuit accommodating unit 4 and the control circuit 6 have the same configuration as those of the first embodiment, and description thereof will not be provided.

The sensor 310 includes a pair of electrodes 320, two terminals 312 and 314, and a fixture 330. The pair of electrodes 320 includes two electrodes 322 and 324. The electrode 322 has the same configuration as the electrode 22 of the first embodiment, and description thereof will not be provided.

The electrode 324 is disposed inside the electrode 322. The electrode 324 has the same configuration as the electrode 224 except that the electrode 324 is formed in a solid columnar shape.

The fixture 330 is formed of resin. The fixture 330 accommodates the pair of electrodes 320, and includes an upper end fixing portion 335, a circumferential wall 336, and a lower end fixing portion 340 similarly to the fixture 230. The upper end fixing portion 335 does not include a thermistor and does not include an accommodating portion and an insertion hole for disposing the thermistor. Besides this, the upper end fixing portion 335 has the same configuration as the upper end fixing portion 235. The terminals 312 and 314 have the same configuration as the terminals 212 and 214, respectively, and description thereof will not be provided.

The circumferential wall 336 and the lower end fixing portion 340 have the same configuration as the circumferential wall 236 and the lower end fixing portion 240, respectively, and description thereof will not be provided. The through-holes 342 and 350 and the base 348, and the intermediate portion 346 have the same configuration as the through-holes 242 and 250, the base 248, and the intermediate portion 246, respectively.

As shown in FIG. 7, the fuel having flowed into the gap between the electrodes 322 and 324 through the through-hole 342 flows toward the through-hole 350 through the slit 322a. According to this configuration, the sensor 310 can provide the same advantages as the sensor 210 of the third embodiment.

### Fifth Embodiment

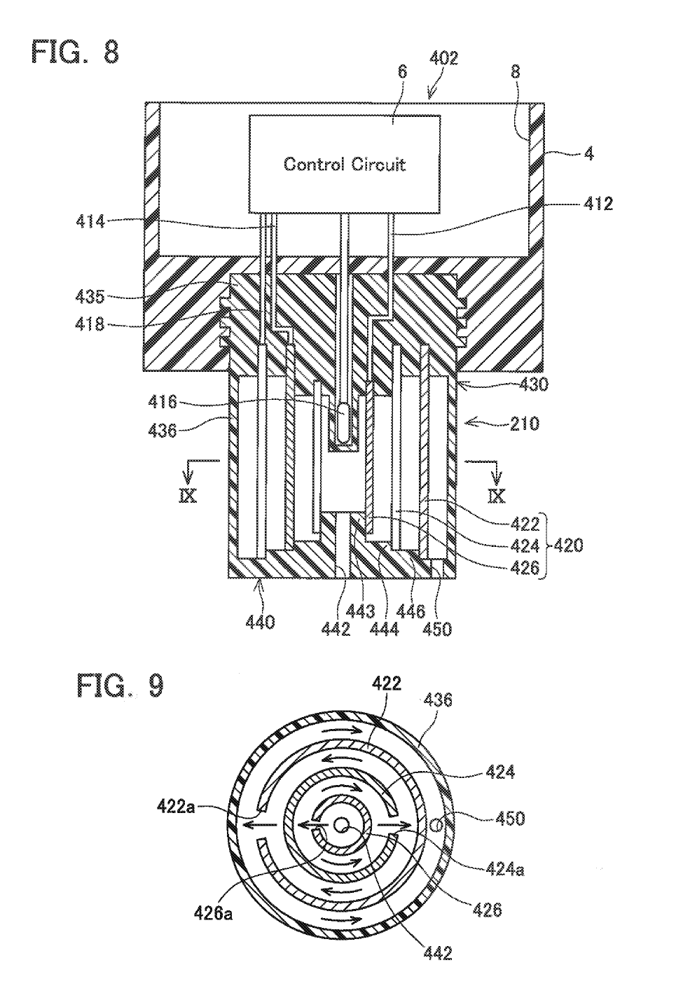

As shown in FIG. 8, a sensor system 402 of a fifth embodiment includes a circuit accommodating unit 4, a control circuit 6, and a sensor 410. The circuit accommodating unit 4 and the control circuit 6 have the same configuration as those of the first embodiment, and description thereof will not be provided.

The sensor 410 includes a set of electrodes 420, three terminals 412, 414, and 418, a thermistor 416, and a fixture 430. The set of electrodes 420 includes three electrodes 422, 424, and 426. The electrodes 424 and 426 have the same configuration as the electrodes 22 and 24, respectively, of the first embodiment, and description thereof will not be provided.

The electrode 422 has a cylindrical shape. The electrode 422 surrounds an outer side of the electrode 424. As shown in FIG. 9, the electrode 422 includes a slit 422a that extends in parallel to an axial direction of the electrode 422. The slit 422a extends from an upper end of the electrode 422 to a lower end thereof. That is, the electrode 422 has a circular-arc shape (C-shape) in which a portion where the slit 422a is formed is cut when seen in a cross-section perpendicular to the axial direction. The slit 422a is formed at such a position that the slit 422a does not face a slit 424a of the electrode 424. The slit 422a is formed on an opposite side (that is, at a position rotated by 180°) from the slit 424a with an axial center of the electrodes 422, 424, 426 interposed. According to this configuration, the slit 422a is formed in the same direction as a slit 426a of the electrode 426 when seen from the axial center.

The fixture 430 is supported by the circuit accommodating unit 4. The fixture 430 is formed of resin. The fixture 430 accommodates the set of electrodes 420. The fixture 430 includes an upper end fixing portion 435, a circumferential wall 436, and a lower end fixing portion 440. The upper end fixing portion 435 covers part of the upper ends of the electrodes 422, 424, and 426. The terminals 412, 414, and 418 are disposed in the upper end fixing portion 435. The terminals 412 and 414 extend from upper ends of the electrodes 424 and 426, respectively, to reach the circuit accommodating chamber 8 while passing through the fixture 30 similarly to the terminals 12 and 14. The terminal 418 extends from an upper end of the electrode 422 to reach the circuit accommodating chamber 8 while passing through the fixture 30. One end (lower end) of the terminal 418 is in contact with the electrode 422, and the other end (upper end) of the terminal 418 is in contact with the control circuit 6.

A cylindrical circumferential wall 436 that extends downward is formed on a lower surface of the upper end fixing portion 435. The circumferential wall 436 extends from the lower surface of the upper end fixing portion 435 up to the same height as a lower end of the electrode 422. The lower end fixing portion 440 is fixed to a lower end of the circumferential wall 436. An upper end of the cylindrical circumferential wall 436 is blocked by the upper end fixing portion 435, and the lower end of the cylindrical circumferential wall 436 is blocked by the lower end fixing portion 440. The lower end fixing portion 440 is in contact with the lower ends of the electrodes 422 to 426. The lower end fixing portion 440 includes a base 448, intermediate portions 446 and 444, and an upper portion 443. Each of the base 448, the intermediate portions 446 and 444, and the upper portion 443 has a cylindrical shape. The lower end of the electrode 422 is in contact with the upper surface of the base 448. The electrodes 424 and 426 are not in contact with the base 448. The intermediate portion 446 protrudes upward from an upper surface of the base 448. The intermediate portion 446 makes contact with an inner circumferential surface of the electrode 422. The intermediate portion 444 protrudes upward from an upper surface of the intermediate portion 446. The intermediate portion 444 makes contact with an inner circumferential surface of the electrode 424. The upper portion 443 protrudes upward from an upper surface of the intermediate portion 444. The upper portion 443 makes contact with an inner circumferential surface of the electrode 426. According to this configuration, it is possible to cause the electrodes 422 to 426 to be strongly aligned with respect to each other. As a result, it is possible to prevent a change in a capacitance of the pair of electrodes 422 and 424 and a capacitance of the pair of electrodes 424 and 426 due to a change in a distance between the electrodes 422 to 426.

A through-hole 450 that communicates with a gap between the electrode 422 and the circumferential wall 436 is formed on the base 448. The through-hole 450 is formed on an opposite side (that is, at a position rotated by 180°) from the slit 422a with the axial center interposed when seen in a cross-section perpendicular to the axial direction of the electrodes 422 to 426. In this configuration, the slit 426a of the electrode 426 and the slit 422a of the electrode 422 are positioned in the same direction about the axial center of the electrodes 422 to 426. Moreover, the slit 424a of the electrode 424 and the through-hole 450 are positioned in the same direction about the axial center of the electrodes 422 to 426 and are formed at a position rotated by 180° from the slits 422a and 426a.

The through-hole 442 is connected to a fuel pump (not shown). As a result, the fuel pressurized by the fuel pump flows into the electrode 426 through the through-hole 442. After that, the fuel flows into the gap between the electrodes 424 and 426 by passing through the slit 426a. The fuel branches into two along an outer circumferential surface of the electrode 426 and flows into a gap between the electrodes 424 and 426. The fuel having reached the slit 424a flows into a gap between the electrodes 422 and 424 by passing through the slit 424a. Then, the fuel branches into two along an outer circumferential surface of the electrode 424 and passes through the gap between the electrodes 422 and 424. Subsequently, the fuel having reached the slit 422a flows into the gap between the electrode 422 and the circumferential wall 436 by passing through the slit 422a. Then, the fuel branches into two along an outer circumferential surface of the electrode 422 and passes through the gap between the electrode 422 and the circumferential wall 436. The fuel having reached the through-hole 450 flows out of the sensor 410 through the through-hole 450. According to this configuration, the sensor 410 can allow the fuel in the gap between the set of electrodes 420 and the circumferential wall 436 to flow smoothly similarly to the sensor 10 of the first embodiment.

The sensor 410 can specify the ethanol concentration in the fuel using the capacitance of the pair of electrodes 422 and 424, the capacitance of the pair of electrodes 424 and 426, and the temperature of the fuel. Thus, it is possible to specify the ethanol concentration with high accuracy as compared to a configuration of specifying the ethanol concentration in the fuel using the capacitance of a pair of electrodes and the fuel temperature.

### Sixth Embodiment

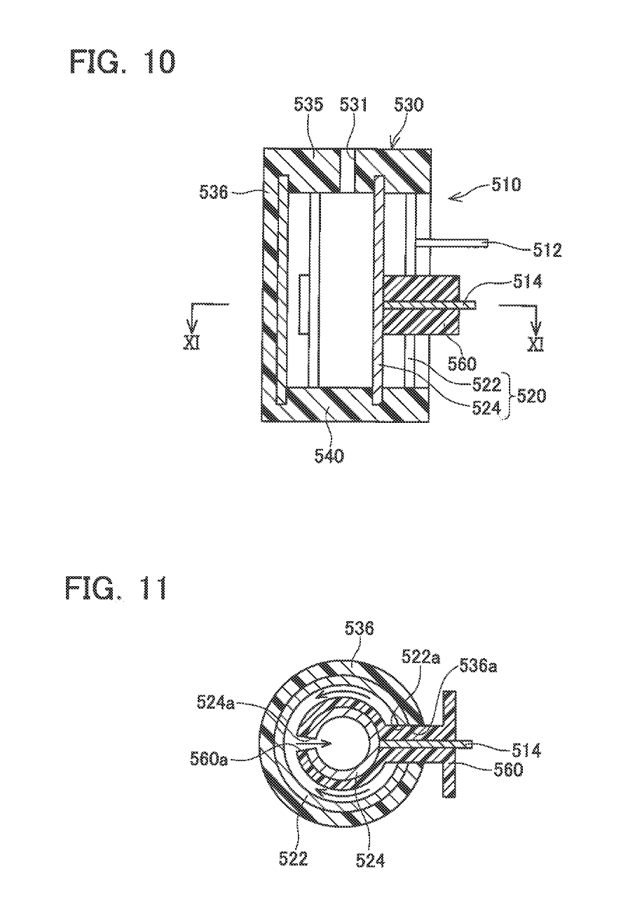

A sensor 510 shown in FIG. 10 includes a pair of electrodes 520, two terminals 512 and 514, and a fixture 530. The pair of electrodes 520 includes two electrodes 522 and 524. The electrodes 522 and 524 have the same configuration as the electrodes 22 and 24, respectively, of the first embodiment, and description thereof will not be provided.

The fixture 530 is formed of resin. The fixture 530 accommodates the pair of electrodes 520. The fixture 530 includes an upper end fixing portion 535, a circumferential wall 536, a lower end fixing portion 540, and an inner electrode fixing portion 560. The circumferential wall 536 covers an outer circumferential surface of the electrode 522. That is, the circumferential wall 536 has a cylindrical shape similarly to the electrode 522 and includes a slit 536a that is formed outside the slit 522a and corresponds to the slit 522a. The circumferential wall 536 makes contact with the outer circumferential surface of the electrode 522 and is fixed to the electrode 522.

An upper end fixing portion 535 is attached to an upper end of the circumferential wall 536. The upper end fixing portion 535 is disposed on upper ends of the electrodes 522 and 524. Part of the upper ends of the electrodes 522 and 524 is inserted in the upper end fixing portion 535. As a result, the upper end fixing portion 535 makes contact with inner circumferential surfaces and outer circumferential surfaces of the electrodes 522 and 524. The upper end fixing portion 535 blocks openings on the upper ends of the electrodes 522 and 524. The upper end fixing portion 535 is connected to the circumferential wall 536 on an outer side of the electrode 522. Moreover, the upper end fixing portion 535 extends from the slit 522a of the electrode 522 toward an outside of the electrode 522 and is connected to the upper end of the circumferential wall 536. A through-hole 531 that passes from the upper surface of the upper end fixing portion 535 to the lower surface is formed at a central portion of the upper end fixing portion 535. Due to the through-hole 531, the outside of the sensor 510 communicates with the inside of the electrode 524.

A lower end fixing portion 540 is connected to a lower end of the circumferential wall 536. The lower end fixing portion 540 is disposed on lower ends of the electrodes 522 and 524. Part of the lower ends of the electrodes 522 and 524 is inserted in the lower end fixing portion 540. As a result, the lower end fixing portion 540 makes contact with the inner circumferential surfaces and the outer circumferential surfaces of the electrodes 522 and 524. The lower end fixing portion 540 blocks openings on the lower ends of the electrodes 522 and 524. The lower end fixing portion 540 is connected to the circumferential wall 536 on the outer side of the electrode 522. Moreover, the lower end fixing portion 540 extends from the slit 522a of the electrode 522 toward the outside of the electrode 522 and is connected to the lower end of the circumferential wall 536. The upper end fixing portion 535 and the lower end fixing portion 540 make contact with both the inner circumferential surfaces and the outer circumferential surfaces of the electrodes 522 and 524. Due to this, the electrodes 522 and 524 are connected by the circumferential wall 536 and are aligned with respect to each other by the upper end fixing portion 535 and the lower end fixing portion 540. In a modification, the upper end fixing portion 535 and the lower end fixing portion 540 may make contact with at least one of the inner circumferential surface and the outer circumferential surface of the electrodes 522 and 524.

The inner electrode fixing portion 560 is disposed at an intermediate position of the circumferential wall 536 (that is, the sensor 510). The inner electrode fixing portion 560 is in contact with the electrode 524 while passing through the slits 522a and 536a from the outside of the circumferential wall 536. The inner electrode fixing portion 560 is fixed to the electrode 524 by surrounding part of the outer circumferential surface of the electrode 524 in the axial direction of the electrode 524. The inner electrode fixing portion 560 has a cylindrical shape similarly to the electrode 524. The inner electrode fixing portion 560 includes a slit 560a that is formed outside the slit 524a of the electrode 524 and corresponds to the slit 524a. The inner electrode fixing portion 560 is fixed to the circumferential wall 536 on the outer side of the electrode 522. Due to this, the electrode 524 is more strongly fixed to the electrode 522 with the inner electrode fixing portion 560 interposed.

The terminal 514 is inserted in the inner electrode fixing portion 560. The terminal 514 is in contact with the electrode 524 by passing through the slits 522a and 536a from the outer side of the circumferential wall 536. One end of the terminal 514 is in contact with the electrode 524, and the other end is in contact with a control circuit (not shown). The terminal 512 is in contact with the electrode 522 by passing through the circumferential wall 536. One end of the terminal 512 is in contact with the electrode 522, and the other end is in contact with the control circuit (not shown).

As shown in FIG. 11, the fuel flows into the gap between the electrodes 522 and 524 by passing through the slit 522a. The fuel having flowed into the gap between the electrodes 522 and 524 branches into two along the outer wall of the inner electrode fixing portion 560 and passes through the gap between the electrodes 522 and 524. With this configuration, it is possible to allow the fuel in the gap between the electrodes 522 and 524 to flow smoothly similarly to the first embodiment.

Moreover, in this configuration, the terminal 514 is brought into contact with the electrode 524 using the slit 522a. Thus, it is not necessary to provide a separate configuration for realizing the contact between the electrode 524 and the terminal 514. Moreover, the inner electrode fixing portion 560 can be brought into contact with the electrode 524 using the slit 522a. Thus, it is not necessary to provide a separate configuration such as a hole for realizing the contact between the electrode 524 and the inner electrode fixing portion 560.

### MODIFICATIONS

(1) In the above embodiments the electrode 22 and the like includes the slit 22a and the like that extends from the upper end of the electrode 22 and the like to the lower end thereof. However, the slit 22a and the like may not extend from the upper end of the electrode 22 and the like to the lower end thereof. For example, in the case of the electrode 22, the slit 22a may be formed in a range where the slit 22a faces the electrode 24. That is, the upper and lower ends of the slit 22a may be closed. In other words, the slit 22a may be formed over an entire length of a range where the electrode 22 faces the electrode 24.

(2) In the above embodiments, the electrode 22 and the like has a cylindrical or columnar shape. However, the electrode 22 and the like may have a polygonal tubular shape or a polygonal columnar shape.

(3) In the above embodiments, the slit 22a and the like are provided in parallel to the axial direction of the electrode 22 and the like. However, the slit 22a and the like may not be in parallel to the axial direction of the electrode 22 and the like. Moreover, the slit 22a and the like may not be linear. Further, the slit 22a and the like may be divided into a plurality of parts. Generally speaking, the slit 22a is formed in a region of the electrode 22 facing directly the electrode 24 so that no continuous surfaces are present when one sees the electrode 22 in a direction perpendicular to the axial direction. In this modification, it can be also said that “a slit is formed so as to communicate between an inside and an outside of a first electrode over an entire length of a range where one of the electrodes faces the other electrode in an axial direction of the first electrode.”

(4) In the first embodiment, the slit 24a is formed on the opposite side (that is, at a position rotated by 180°) from the slit 22a with the axial center interposed. However, the slit 24a may be formed at a position rotated by a predetermined angle (for example, 45°, 90°, or the like) from the axial center of the electrodes 22 and 24 and the slit 22a. In other words, a direction extending from the axial center of the electrodes 22 and 24 toward the central position of the width (the length in the circumferential direction of the electrode 22) of the slit 22a may be different from a direction extending from the axial center of the electrodes 22 and 24 toward the central position of the width (the length in the circumferential direction of the electrode 24) of the slit 24a. This modification also falls within the configuration “the second slit is formed at such a position that the second slit does not face the first slit.”

(5) The sensor of the present description can be used for detecting a liquid level of fuel, the temperature of fuel, or the like in addition to the ethanol concentration in the fuel. Alternatively, the sensor of the present description can be used for detecting the quality (for example, concentration of a specific substance included in a liquid) of a liquid (for example, a solution) other than fuel, a liquid level of a liquid other than fuel, the temperature of a liquid other than fuel, and the like.

What is claimed is:

1. A sensor comprising:
a first tubular electrode; and
a second electrode disposed within the first electrode so as to face an inner circumferential surface of the first electrode, wherein
a slit is formed on one of or both of the first and second electrodes so as to communicate between an inside and an outside of the electrode on which the slit is formed over an entire length of a range where the electrode on which the slit is formed faces the other electrode in an axial direction of the first electrode.

2. The sensor according to claim 1, wherein
the second electrode has a tubular shape,
the slit is formed on each of the first and second electrodes, the slit including a first slit and a second slit,
the first slit is formed on the first electrode over an entire length of a range where the first electrode faces the second electrode in the axial direction of the first electrode,
the second slit is formed on the second electrode over an entire length of a range where the second electrode faces the first electrode in the axial direction of the first electrode, and
the second slit is formed at such a position that the second slit does not face the first slit.

3. The sensor according to claim 2, wherein
the second electrode has a tubular shape,
the sensor further includes a third tubular electrode disposed within the second electrode so as to face an inner circumferential surface of the second electrode, and
a third slit is formed on the third electrode over an entire length of a range where the third electrode faces the second electrode in the axial direction of the first electrode.

4. The sensor according to claim 1, further comprising:
a terminal connected to the second electrode, wherein
the slit is formed on the first electrode over an entire length of a range where the first electrode faces the second electrode in the axial direction of the first electrode, and
the terminal passes through the slit from the outside of the first electrode and is connected to the second electrode.

5. The sensor according to claim 1, further comprising:
a fixture that fixes the second electrode to the first electrode, wherein
the slit is formed on the first electrode over an entire length of a range where the first electrode faces the second electrode in the axial direction of the first electrode, and
the fixture passes through the slit from the outside of the first electrode and makes contact with the second electrode.

6. The sensor according to claim 1, further comprising:
a fixture attached to both ends of the first and second electrodes in the axial direction so as to fix the second electrode to the first electrode.

7. The sensor according to claim 6, further comprising:
a terminal connected to the first and second electrodes, wherein
the terminal passes through the fixture from a surface of the fixture opposite to the first electrode and is connected to the first and second electrodes.

8. The sensor according to claim 6, wherein
the second electrode has a tubular shape, and
the sensor further includes a temperature sensor supported by the fixture and accommodated inside the second electrode.
